(19) United States
(12) Patent Application Publication
(10) Pub. No.: US 2026/0348991 A1
(43) Pub. Date: Nov. 5, 2026

(54) COMPUTER IMPLEMENTED METHOD FOR QUALITY ASSURANCE OF AN IN VITRO-DIAGNOSTIC ANALYZER

(71) Applicants: F. HOFFMANN-LA ROCHE AG, Basel (CH); Hitachi High-Tech Corporation, Tokyo (JP)

(72) Inventors: Mohamed Abouelsoud, Rotkreuz (CH); Elisabeth Foester, Penzberg (DE); Teruhiro Yamano, Tokyo (JP); Satoshi Shibuya, Tokyo (JP); Toma Onose, Tokyo (JP)

(73) Assignees: F. HOFFMANN-LA ROCHE AG, Basel (CH); Hitachi High-Tech Corporation, Tokyo (JP)

(21) Appl. No.: 19/486,463

(22) PCT Filed: May 21, 2024
(86) PCT No.: PCT/EP2024/063963

§ 371 Date: Nov. 21, 2025

(30) Foreign Application Priority Data
May 22, 2023 (EP) 23174520.9

(51) Int. Cl. G01N 35/00

(52) U.S. Cl. CPC G01N 35/00613

(57) ABSTRACT

A computer implemented method for quality assurance of an in vitro-diagnostic analyzer is proposed. The method comprising
a) retrieving at least one recall message comprising information about an identity of a recalled item of the analyzer;
b) processing the recall message by determining if the recalled item is onboard the analyzer;
c) applying, in case the recalled item is determined to be onboard the analyzer at least one quality assurance action on the analyzer.

### TECHNICAL FIELD

The invention refers to a computer implemented method for quality assurance of an in vitro-diagnostic analyzer, an in vitro-diagnostic analyzer for analyzing at least one sample, a computer program, a computer-readable storage medium and a non-transient computer-readable medium. The method and device according to the present invention may be used laboratories comprising in vitro-diagnostic analyzers. Other applications are generally feasible.

### BACKGROUND ART

Items of or used by in vitro-diagnostic analyzers such as consumable, reagent, instrument components, spare parts used on the in vitro-diagnostic analyzer may be object of a recall due to quality issues. In this case, usually, the user is informed of the recall and supply of replacement material is assured. However, in case there is an item to be recalled present at the in vitro-diagnostic analyzer, there is no possibility to block the use of that item. Thus, users can still use the item to be recalled and can produce test results, even after they have been informed about the recall. This can result in non-reliable and wrong test results.

US20010056359A1 describes an automated system and method for communicating product information to consumers through a central computer using a distributed computer network. The central computer is connected to a plurality of public health and product recall information sources through a public network such as the Internet. Consumers purchasing or otherwise linked with products submit product information to the central computer. The central computer establishes a link between the user and associated product information and stores the data in a database. User medical information is also submitted to and retained by the central computer. Upon receipt of product update information, which may include recall notification or other product warnings from the public health or product recall information sources, the central computer accesses the database and identifies user-product links impacted by the product update information and automatically initiates electronic or other communications to affected users. Similarly, users purchasing products can access the central computer and search for stored product information which may impact the purchasing decision. Medical treatments may also be verified against information stored in the central computer pertaining to user health conditions, other treatments currently being undertaken, etc. to avoid dangerous interactions.

JP05808582B2 describes an autoanalyzer which includes: an analysis part which analyzes a sample; a display part which displays information about analysis of the sample; and a control part which controls the analysis part to perform analysis processing of the sample. The control part displays a picture showing information on a reagent to be used in analysis of the sample on the display part, uses a mask instruction of the reagent operated based on the display picture as input, performs mask designation of the reagent on the basis of the input mask instruction of the reagent, and does not use the mask designated reagent in the analysis of the sample.

US 2019/212353 A describes a detection device and a detection method. The detection device comprises: a consumable cassette configured to have a detection side provided with a consumable, the consumable cassette being provided with a circuit board carrying a test information; and a detection instrument configured to read the test information from the circuit board and perform a detection to the consumable based on the test information to obtain a detection result.

US 2017/032091 A1 describes a method of providing dynamic analysis for troubleshooting in vitro diagnostics instrument issues which includes receiving, at a second computing device in communication with a plurality of instruments, identification of an issue associated with a portion of an instrument of the plurality of instruments, the identification received from a first computing device in communication with the instrument. A central computing device accesses data from one or more databases and determines an ordering of one or more corrective actions for resolving the issue by applying the data to a probabilistic model based on at least one of: patterns from the plurality of instruments; and operator input. The central computing device provides the one or more corrective actions in the determined order to the first computing device to be displayed via a user interface at the instrument.

US 2020/320198 A1 describes an example device which comprises a plurality of components and a BIOS which include a non-volatile memory having instructions stored thereon that are executable by a processor of the device to cause the BIOS to determine a component identification (ID) of a component of the plurality of components, and fetch a plurality of recall component IDs. The instructions also are to cause the BIOS to compare the determined component ID with the plurality of recall component IDs. In response to a correspondence between the determined component ID and one recall component ID of the plurality of component IDs, the BIOS is to transmit signals representing a prompt to place the component of the plurality of components in a safety mode. The instructions also cause the BIOS to receive signals in response to the prompt and, responsive to the received signals, disable the component of the plurality of components.

### Problem to be Solved

It is therefore an objective of the present invention to provide a computer implemented method for quality assurance of an in vitro-diagnostic analyzer, an in vitro-diagnostic analyzer for analyzing at least one sample, a computer program, a computer-readable storage medium and a non-transient computer-readable medium which at least partially avoid the shortcomings of known devices and methods of this kind and which at least partially address the abovementioned challenges. Specifically, devices and methods shall be disclosed which allow quality assurance of an in vitro-diagnostic analyzer.

### SUMMARY

This problem is addressed by a computer implemented method for quality assurance of an in vitro-diagnostic analyzer, an in vitro-diagnostic analyzer for analyzing at least one sample, a computer program, a computer-readable storage medium and a non-transient computer-readable medium with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a computer implemented method for quality assurance of an in vitro-diagnostic analyzer is disclosed.

The term “computer implemented method” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one processor which is configured for performing at least one of the method steps of the method according to the present invention. Preferably each of the method steps is performed by the computer and/or computer network. The method may be performed completely automatically, specifically without user interaction. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user. The method may be performed software-mediated.

The term “in vitro-diagnostics (IVD) analyzer”, also denoted as “analyzer” herein, as used herein is a broad term and is to be given its ordinary and customary meaning to a person of ordinary skill in the art and is not to be limited to a special or customized meaning. The term specifically may refer, without limitation, to an analyzer configured for performing at least one test on at least one sample, e.g. a biological sample that have been taken off the human body or animal body, in particular a plurality of samples. The samples to be analyzed may be prepared for the analysis using at least one reagent. For example, the test on the sample may comprise applying at least one reagent to the sample and monitoring a detectable reaction. For example, the IVD analyzer may be part of an IVD laboratory such as a chemical laboratory, a clinical laboratory, a medical laboratory, a forensic laboratory or a blood bank. For example, the IVD analyzer is used for electrochemical and/or spectroscopic experiments. The IVD analyzer may be configured for executing one or more processing steps and/or workflow steps on one or more biological samples and/or reagents. The processing step may be physically executed processing steps such as centrifugation, aliquotation, sample analysis and the like. The IVD analyzer may be and/or may comprise pre-analytical sample work-cells, post-analytical sample workcells and also analytical work-cells. The IVD analyzer may be one or more of a pre-analytical, an analytical, or a post-analytical instrument. For example, the processing steps may be pre-analytical processing steps (such as aliquoting, sample preparation), analytical processing steps (such as an assay to determine the presence and/or concentration of an analyte in the biological sample) or post-analytical processing steps (such as archiving of the biological sample). A pre-analytical instrument can usually be used for the preliminary processing of samples or sample vessels. An analytical instrument can be designed, for example, to use a sample or part of the sample and a reagent in order to produce a measurable signal, on the basis of which it is possible to determine whether the analyte is present, and if desired in what concentration. A post-analytical instrument can usually be used for the post-processing of samples like the archiving of samples. The IVD analyzer may comprise a pipetting device for pipetting a sample and further devices such as a sorting device for sorting samples or sample vessels, a cap removal device for removing caps or closures on sample vessels, a cap fitting device for fitting caps or closures on sample vessels, an aliquoting device for aliquoting samples, a centrifuging device for centrifuging samples, an analyzing device for analyzing a sample, a heating device for heating a sample, a cooling device for cooling a sample, a mixing device for mixing a sample, a separation device for isolating an analyte of the sample, a storing device for storing samples, an archiving device for archiving samples, a sample vessel type determination device for determining a sample vessel type, a sample quality determination device for determining a sample quality.

The IVD analyzer may be configured to obtain at least one measurement value. For example, the IVD analyzer may be operable for determining via various chemical, biological, physical, optical or other technical procedures a parameter value of the sample or a component thereof. For example, the IVD analyzer may be operable for measuring said parameter of the sample or of at least one analyte and return the obtained measurement value. The list of possible analysis results returned by the IVD analyzer may comprise, without limitation, concentrations of the analyte in the sample, a digital (yes or no) result indicating the existence of the analyte in the sample (corresponding to a concentration above the detection level), optical parameters, DNA or RNA sequences, data obtained from mass spectroscopy of proteins or metabolites and physical or chemical parameters of various types. For example, the IVD analyzer may comprise units assisting with the pipetting, dosing, and mixing of samples and/or reagents. The IVD analyzer may comprise a reagent holding unit for holding reagents to perform the assays. Reagents may be arranged for example in the form of vessels, containers or cassettes containing individual reagents or group of reagents, placed in appropriate receptacles or positions within a storage compartment or conveyor. The IVD analyzer may comprise a consumable feeding unit. The IVD analyzer may comprise a process and detection system whose workflow is optimized for certain types of analysis. Examples of such IVD analyzer are clinical chemistry analyzers, coagulation chemistry analyzers, immunochemistry analyzers, urine analyzers, nucleic acid analyzers, used to detect the result of chemical or biological reactions or to monitor the progress of chemical or biological reactions.

For example, the IVD analyzer may be a pre-analytical laboratory instrument. The pre-analytical laboratory instrument may be or may comprise one or more devices for executing one or more pre-analytical container-processing steps on one or more sample container such as on a sample contained by a sample container. The pre-analytical laboratory instrument may be configured for preparing the sample container, e.g. a sample contained by the sample container, for one or more succeeding analytical tests. For example, a pre-analytical container-processing step can be, for example, a centrifugation step, a capping-, decapping- or recapping step, an aliquotation step, a step of adding buffers to a sample and the like. The pre-analytical laboratory instrument may be configured for pretreating the biological sample prior to use, such as preparing plasma or serum from blood, diluting viscous fluids, lysis or the like. The pretreating may involve filtration, distillation, concentration, inactivation of interfering components, and the addition of reagents. The pre-analytical laboratory instrument may be configured for rendering an initially solid or semi-solid biological sample liquid by dissolving or suspending it with a suitable liquid medium.

The term “sample” as used herein is a broad term and is to be given its ordinary and customary meaning to a person of ordinary skill in the art and is not to be limited to a special or customized meaning. The term specifically may refer, without limitation, to a material suspected of containing an analyte of interest. The sample can be derived from any biological source, such as a physiological fluid, including, blood or blood derivatives such as plasma or serum, saliva, ocular lens fluid, cerebral spinal fluid, sweat, urine, milk, ascites fluid, mucous, synovial fluid, peritoneal fluid, amniotic fluid, tissue, cells or the like. The sample can be pretreated prior to use, such as preparing plasma or serum from blood, diluting viscous fluids, lysis or the like; methods of treatment can involve filtration, distillation, concentration, inactivation of interfering components, and the addition of reagents. A sample may be used directly as obtained from the source or following a pretreatment to modify the character of the sample, e.g. after being diluted with another solution or after having being mixed with reagents e.g. to carry out one or more diagnostic assays like e.g. clinical chemistry assays, immunoassays, coagulation assays, nucleic acid testing, etc. The term “sample” as used herein is therefore not only used for the original sample but also relates to a sample which has already been processed such as pipetted, diluted, mixed with reagents, enriched, having been purified, having been amplified and the like. As used herein, the term “analyte” may refer to the compound or composition to be detected or measured. The IVD analyzer may be configured for processing the sample. The term “processing a sample” as used herein is a broad term and is to be given its ordinary and customary meaning to a person of ordinary skill in the art and is not to be limited to a special or customized meaning. The term specifically may refer, without limitation, to any treatment of the sample. For example, the processing may comprise one or more of transferring, aliquoting, isolating, purifying, incubating, reacting a sample or combining a reagent with a sample. The term “reagent” as used herein is a broad term and is to be given its ordinary and customary meaning to a person of ordinary skill in the art and is not to be limited to a special or customized meaning. The term specifically may refer, without limitation, to a composition required for treatment of a sample. Reagents may be any liquid, e.g. a solvent or chemical solution, which needs to be mixed with a sample and/or other reagent in order e.g. for a reaction to occur, or to enable detection. A reagent may be for example a diluting liquid, including water, it may comprise an organic solvent, it may comprise a detergent, it may be a buffer. Reagents may also be dry reagents adapted e.g. to be dissolved by a sample, another reagent or a diluting liquid. A reagent in the more strict sense of the term may be a liquid solution containing a reactant, typically a compound or agent capable e.g. of binding to or chemically transforming one or more analytes present in a sample. Examples of reactants are enzymes, enzyme substrates, conjugated dyes, protein-binding molecules, nucleic acid binding molecules, antibodies, chelating agents, promoters, inhibitors, epitopes, antigens, etc.

For example, the IVD analyzer may be a post-analytical laboratory instrument. The post-analytical laboratory instrument may be configured for performing at least one post-analytical container-processing step. The post-analytical laboratory instrument may be configured for automatically processing and/or storing one or more samples. For example, the post-analytical container-processing steps may comprise a recapping step, a step for unloading a sample from the sample container or a step for transporting the sample container to a storage unit or to a unit for collecting biological waste.

The term “quality assurance” as used herein is a broad term and is to be given its ordinary and customary meaning to a person of ordinary skill in the art and is not to be limited to a special or customized meaning. The term specifically may refer, without limitation, to ensuring one or more of reliability, confidence and credibility of measurement results within defined tolerances, e.g. that measurement results are valid with respect to one or more of traceability, accuracy, and precision. Specifically, the quality assurance may comprise blocking the generation of results in order not to generate results that maybe not reliable. The quality assurance may allow generating results but with attaching a flag to indicate that the result maybe not reliable.

The method steps may be performed in the given order or may be performed in a different order. Further, one or more additional method steps may be present which are not listed. Further, one, more than one or even all of the method steps may be performed repeatedly. The method comprises the following steps:
a) retrieving at least one recall message comprising information about an identity of a recalled item of the analyzer;
b) processing the recall message by determining if the recalled item is onboard the analyzer;
c) applying, in case the recalled item is determined to be onboard the analyzer, at least one quality assurance action on the analyzer.

The term “retrieve” as used herein is a broad term and is to be given its ordinary and customary meaning to a person of ordinary skill in the art and is not to be limited to a special or customized meaning. The term specifically may refer, without limitation, to the process of obtaining data from at least one data source. The data source may, however, vary, in accordance with the specific application. For example, the retrieving of the at least one recall message may take place by at least one of the following: downloading the recall message such as from at least one data storage device, from a web-or cloud-based data storage device; obtaining the recall message via at least one computer network, such as the Internet; obtaining the recall message via at least one wire-based and/or wireless interface. The retrieving may fully or partially take place automatically, such as by automatic download of the recall message. The retrieving may comprise, e.g. in addition, manual steps, e.g. as a part of a service/maintenance workflow such as inputting recall files.

For example, the recall message is retrieved via at least one communication interface of the IVD analyzer. The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. The communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may provide means for transferring or exchanging information. The communication interface may provide a data transfer connection, e.g. Bluetooth, NFC, inductive coupling or the like. As an example, the communication interface may be or may comprise at least one port comprising one or more of a network or internet port, a USB-port and a disk drive. The communication interface may further comprise at least one display device. The communication interface may be at least one web interface.

For example, the recall message may be retrieved automatically, e.g. is retrieved automatically via the communication interface configured for retrieving. For example, the retrieving of the recall message may be automatically executed at predefined time intervals. For example, the recall message may be sent remotely to the analyzer. The predefined time intervals may be adapted to the routines of the system, e.g. synchronization to a repository may take place every 24 hours. No action may be required on the part of the customer/user.

The term “recall” as used herein is a broad term and is to be given its ordinary and customary meaning to a person of ordinary skill in the art and is not to be limited to a special or customized meaning. The term specifically may refer, without limitation, to a request from a manufacturer to return at least one item of the analyzer or to a request not to use that item any longer and/or to replace that item (denoted as recalled item herein), e.g. after a discovery of malfunction and/or defects and/or safety issues. The recalled item may be any item directly or indirectly used for generating the measurement result. The recalled item may be any item which is administered in a database and/or software by using an identifier. The recalled item, for example, may be at least one item selected from the group comprising: at least one reagent; at least one diluent; at least one detergent; at least one consumable, e.g. pipette tip, assay cup or reaction vessel, or consumable rack or tray, at least one ion selective electrode, at least one spare part, assay reagent, diluent, detergent, system reagent, calibrator, control sample, assay application, calibrator application, control application, electrodes, software versions and the like.

The term “recall message” as used herein is a broad term and is to be given its ordinary and customary meaning to a person of ordinary skill in the art and is not to be limited to a special or customized meaning. The term specifically may refer, without limitation, to a computer readable file, e.g. of an arbitrary computer readable data format such as an xml file or HL7, comprising information about at least one recall, specifically about the identity of the recalled item. The term “information about an identity of a recalled item of the analyzer” as used herein is a broad term and is to be given its ordinary and customary meaning to a person of ordinary skill in the art and is not to be limited to a special or customized meaning. The term specifically may refer, without limitation, to any information allowing for unique identification of an item of the analyzer. The information about the identity of the recalled item may comprise one or more of at least one identifier, a code, a lot number, a version number, information about affected analyzer, information about a compartment ID, an affected software version, time limit (e.g. after X hours), and information about an action to be applied. Other information may be provided by the recall message, too, such as a file expiration date, and the like. The “code” may be a reagent code or application code. Each test application may require an own application code and reagent code. In particular, because reagents are usually produced in several lots. For example, the recall message may comprise a recall file which comprises information about the identity of the item to be recalled, e.g. one or more of its code, its lot number and version number, the affected software version and, optionally at least one action to be applied on the IVD analyzer and/or the laboratory comprising the IVD analyzer.

The term “onboard” as used herein is a broad term and is to be given its ordinary and customary meaning to a person of ordinary skill in the art and is not to be limited to a special or customized meaning. The term specifically may refer, without limitation, to physically present at the analyzer, e.g. as a component or element of the analyzer or used by the analyzer such as a reagent, and/or non-physical functional components, e.g. software, used by the analyzer. Thus, step b) may comprise checking if the recalled item is available on the IVD analyzer and/or if the currently installed software affected.

For example, subsequent to receiving the recall message in step a) and/or during receiving of the recall message, the method further may comprise displaying at least one information about the retrieving of the recall message and/or about the recall message via at least one user interface, e.g. at least one graphical user interface (GUI), e.g. to inform the user on the GUI about the receiving of a recall file.

The recall message is processed by determining if the recalled item is onboard the analyzer. The term “to process the recall message” as used herein is a broad term and is to be given its ordinary and customary meaning to a person of ordinary skill in the art and is not to be limited to a special or customized meaning. The term specifically may refer, without limitation, to at least one arbitrary action/process such as a workflow on the recall message for determining if the recalled item is onboard the analyzer.

The processing of the recall message may comprise reading the recall message. The processing of the recall message may comprise searching with respect to the information about the identity of the recalled item in at least one database of the analyzer and/or in at least one remote database. The term “database” as used herein is a broad term and is to be given its ordinary and customary meaning to a person of ordinary skill in the art and is not to be limited to a special or customized meaning. The term specifically may refer, without limitation, to an arbitrary collection of information, such as information stored in at least one data storage device. The database may comprise the at least one data storage device with the information stored therein. The database may contain an arbitrary collection of information. The database may be or may comprise at least one database selected from the group comprising: at least one server, at least one server system comprising a plurality of servers, at least one cloud server or cloud computing infrastructure. The database may comprise at least one storage unit configured to store data. The determining if the recalled item is onboard the analyzer further may comprise determining if an installed software is affected.

The processing may be performed by using at least one processor. The term “processor” or “processing device” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. The processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. The processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming, for performing one or more evaluation operations.

The processing may be triggered, e.g. automatically, in response to receiving the recall message.

The method may further comprise detecting that a recalled item is registered at a later time point. For example, in case a new item is registered, the method may comprise searching with respect to the identity of the new item in the database of the analyzer and/or in the remote database and may determine if the new item is object to a recall. In this case, at least one quality assurance action may be applied. Otherwise, e.g. in case the new item is not determined to be object to a recall, operation of the analyzer may be continued.

Step c) comprises applying, in case the recalled item is determined to be onboard the analyzer, at least one quality assurance action on the analyzer.

The term “quality assurance action” as used herein is a broad term and is to be given its ordinary and customary meaning to a person of ordinary skill in the art and is not to be limited to a special or customized meaning. The term specifically may refer, without limitation, to at least one action having an impact on the quality assurance. For example, the quality assurance action may comprise at least one action selected from the group comprising: displaying at least one information about presence of the recalled item onboard the analyzer; preventing usage of the recalled item and/or related to the recalled item for further operation of the analyzer by one or more of automatically masking the recalled item in a control menu and/or preventing execution of software relating to the recalled item; preventing operation of the analyzer; blocking at least one affected module and/or at least one affected unit; applying at least one action as defined in the recall message such as attaching a data alarm to every result which is produced by using the recalled item. With respect to blocking at least one affected module and/or at least one affected unit, the IVD analyzer can be designed modular, e.g. several identical modules (e.g. AAA) or different ones (e.g. ABC) or a mix (e.g. AABC) can be combined in one system. Each module can relate to one detection technology having its specific hardware. The hardware of the module can be connected via a core unit and can be controlled by a core software. Alternatively, a plurality of detection technologies may be integrated in one module, resulting in having one hardware with different units. Thus, the quality assurance action may comprise blocking at least one affected module and/or at least one affected unit depending on the design of the IVD analyzer.

For example, the quality assurance action may comprise to either block the use of the recalled item, e.g. in case of an assay reagent, or to block the affected module or unit, e.g. in case of a system reagent or spare part or consumable, or the block the whole system in case the whole system is affected, or to apply an action as defined in the recall file, e.g. item can still be used, but a data alarm is attached to every result which is produced by using the recalled item. The use of a recalled item is safely blocked, or allowed only under defined conditions.

The method may comprise unblocking, e.g. by the control unit, one or more of the recalled item, the affected unit, the affected module or the analyzer, when the recalled item is re-moved from the analyzer and/or exchanged with a not affected one and/or after receiving a message indicating that the item can be used again such as after a software update.

In case the recalled item is determined not to be onboard the analyzer, operation of the analyzer may be continued and/or at least one information about the absence of the recalled item onboard the analyzer may be displayed.

For example, if an item, e.g. consumable, reagent, instrument component used on the IVD analyzer is object of a recall, a recall message is sent remotely to the analyzer. If the item may be found to be onboard confirming that the analyzer is affected, the item is automatically masked and any operation related to that item is prevented until the issue related to the item is resolved.

In a further aspect, an in vitro-diagnostic analyzer for analyzing at least one sample is disclosed. The analyzer comprises
at least one communication interface configured for retrieving at least one recall message comprising information about an identity of a recalled item of the analyzer;
at least one processor configured for processing the recall message by determining if the recalled item is onboard the analyzer;
at least one control unit configured for applying, in case the recalled item is determined to be onboard the analyzer, at least one quality assurance action on the analyzer.

The analyzer may be configured for performing the method according to the present invention. For details, options and definitions, reference may be made to the method as discussed above.

The term “control unit” as used herein is a broad term and is to be given its ordinary and customary meaning to a person of ordinary skill in the art and is not to be limited to a special or customized meaning. The term specifically may refer, without limitation, to an arbitrary device or system configured for performing the named operations, preferably by using at least one data processing device and, more preferably, by using at least one processor and/or at least one application-specific integrated circuit. Thus, as an example, the at least one control unit may comprise at least one data processing device having a software code stored thereon comprising a number of computer commands. The control unit may provide one or more hardware elements for performing one or more of the named operations and/or may provide one or more processors with software running thereon for performing one or more of the named operations. The control unit may comprise one or more programmable devices such as one or more computers, application-specific integrated circuits (ASICs), Digital Signal Processors (DSPs), or Field Programmable Gate Arrays (FPGAs). Additionally or alternatively, however, the control unit may also fully or partially be embodied by hardware.

Further disclosed and proposed herein is a computer program including computer-executable instructions for performing the method according to the present invention in one or more of the embodiments enclosed herein when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps a) to c) as indicated above may be performed by using a computer or a computer network, preferably by using a computer program.

Further disclosed and proposed herein is a computer program product having program code means, in order to perform the method according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a non-transient computer-readable medium including instructions that, when executed by one or more processors, cause the one or more processors to perform the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network. Finally, disclosed and proposed herein is a modulated data signal which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements. Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing and without excluding further possible embodiments, the following embodiments may be envisaged:

Embodiment 1. A computer implemented method for quality assurance of an in vitro-diagnostic analyzer, the method comprising
a) retrieving at least one recall message comprising information about an identity of a recalled item of the analyzer;
b) processing the recall message by determining if the recalled item is onboard the analyzer;
c) applying, in case the recalled item is determined to be onboard the analyzer, at least one quality assurance action on the analyzer.

Embodiment 2. The method according to the preceding embodiment, wherein the quality assurance action comprises at least one action selected from the group comprising: displaying at least one information about presence of the recalled item onboard the analyzer; preventing usage of the recalled item and/or related to the recalled item for further operation of the analyzer by one or more of automatically masking the recalled item in a control menu and/or preventing execution of software relating to the recalled item; preventing operation of the analyzer; blocking at least one affected module and/or at least one affected unit; applying at least one action as defined in the recall message such as attaching a data alarm to every result which is produced by using the recalled item.

Embodiment 3. The method according to the preceding embodiment, wherein the method comprises unblocking one or more of the recalled item, the affected unit, the affected module or the analyzer, when the recalled item is removed from the analyzer and/or exchanged with a not affected one and/or after receiving a message indicating that the item can be used again such as after a software update.

Embodiment 4. The method according to any one of the preceding embodiments, wherein the method comprises detecting that a recalled item is registered at a later time point and to apply at least one quality assurance action.

Embodiment 5. The method according to any one of the preceding embodiments, wherein, in case the recalled item is determined not to be onboard the analyzer, operation of the analyzer is continued and/or at least one information about the absence of the recalled item onboard the analyzer is displayed.

Embodiment 6. The method according to any one of the preceding embodiments, wherein the recalled item is at least one item selected from the group comprising: at least one reagent; at least one diluent; at least one detergent; at least one consumable, e.g. pipette tip, assay cup or reaction vessel, or consumable rack or tray, at least one ion selective electrode, at least one spare part, assay reagent, diluent, detergent, system reagent, calibrator, control sample, assay application, calibrator application, control application, electrodes, software versions and the like.

Embodiment 7. The method according to any one of the preceding embodiments, wherein the recall message is retrieved via at least one communication interface of the analyzer.

Embodiment 8. The method according to any one of the preceding embodiments, wherein the recall message is retrieved automatically.

Embodiment 9. The method according to any one of the preceding embodiments, wherein the retrieving of the recall message is automatically executed at predefined time intervals.

Embodiment 10. The method according to any one of the preceding embodiments, wherein the recall message is sent remotely to the analyzer.

Embodiment 11. The method according to any one of the preceding embodiments, wherein the method comprises displaying at least one information about the retrieving of the recall message and/or about the recall message via at least one user interface, e.g. at least one graphical user interface (GUI).

Embodiment 12. The method according to any one of the preceding embodiments, wherein the information about the identity of the recalled item comprises one or more of at least one identifier, a code, a lot number, a version number, information about affected analyzer, information about a compartment ID, an affected software version, time limit, and information about an action to be applied.

Embodiment 13. The method according to any one of the preceding embodiments, wherein the determining if the recalled item is onboard the analyzer comprises searching with respect to the information about the identity of the recalled item in at least one database of the analyzer and/or in at least one remote database.

Embodiment 14. The method according to any one of the preceding embodiments, wherein the determining if the recalled item is onboard the analyzer comprises determining if an installed software is affected.

Embodiment 15. An in vitro-diagnostic analyzer for analyzing at least one sample, the analyzer comprises
at least one communication interface configured for retrieving at least one recall message comprising information about an identity of a recalled item of the analyzer;
at least one processor configured for processing the recall message by determining if the recalled item is onboard the analyzer;
at least one control unit configured for applying, in case the recalled item is determined to be onboard the analyzer, at least one quality assurance action on the analyzer.

Embodiment 16. The analyzer according to the preceding embodiment, wherein the analyzer is configured for performing the method according to any one of the preceding embodiments referring to a method.

Embodiment 17. A computer program comprising instructions which, when the program is executed by an in vitro-diagnostic analyzer according to any one of the preceding embodiments referring to an analyzer, cause the analyzer to perform the method according to any one of the preceding embodiments referring to a method.

Embodiment 18. A computer-readable storage medium comprising instructions which, when the instructions are executed by the an in vitro-diagnostic analyzer according to any one of the preceding embodiments referring to an analyzer, cause the analyzer to perform the method according to any one of the preceding embodiments referring to a method.

Embodiment 19. A non-transient computer-readable medium including instructions that, when executed by one or more processors, cause the one or more processors to perform the method according to any one of the preceding embodiments referring to a method.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

### In the Figures:

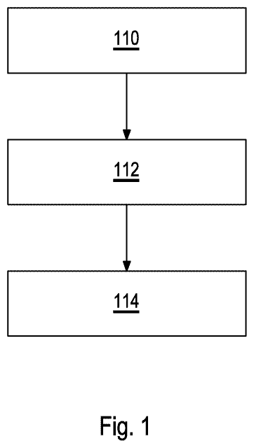

FIG. 1 shows a flowchart of an embodiment of a computer implemented method for quality assurance according to the present invention; and

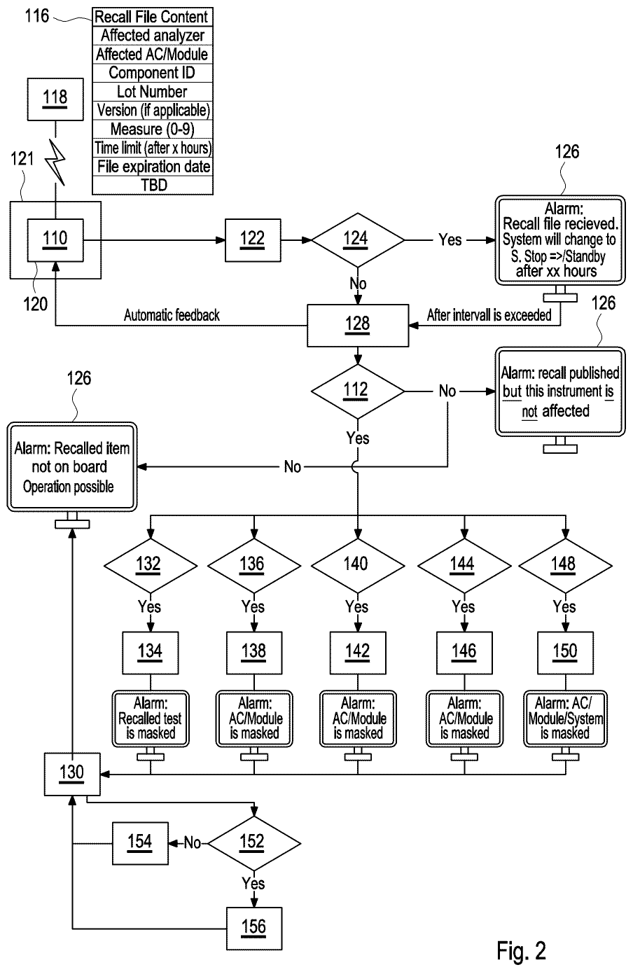

FIG. 2 shows a flowchart of an exemplary embodiment of the method for quality assurance according to the present invention.

### DETAILED DESCRIPTION OF THE EMBODIMENTS

FIG. 1 shows a flowchart of an embodiment of a computer implemented method for quality assurance of an IVD analyzer 121 according to the present invention.

For example, the IVD analyzer 121 may be part of an IVD laboratory such as a chemical laboratory, a clinical laboratory, a medical laboratory, a forensic laboratory or a blood bank. For example, the IVD analyzer 121 is used for electrochemical and/or spectroscopic experiments. The IVD analyzer 121 may be configured for executing one or more processing steps and/or workflow steps on one or more biological samples and/or reagents. For example, the sample can be derived from any biological source, such as a physiological fluid, including, blood or blood derivatives such as plasma or serum, saliva, ocular lens fluid, cerebral spinal fluid, sweat, urine, milk, ascites fluid, mucous, synovial fluid, peritoneal fluid, amniotic fluid, tissue, cells or the like.

The method steps may be performed in the given order or may be performed in a different order. Further, one or more additional method steps may be present which are not listed. Further, one, more than one or even all of the method steps may be performed repeatedly. The method comprises the following steps:
a) (reference number 110) retrieving at least one recall message 116 comprising information about an identity of a recalled item of the analyzer 121;
b) (reference number 112) processing the recall message 116 by determining if the recalled item is onboard the analyzer 121;
c) (reference number 114) applying, in case the recalled item is determined to be onboard the analyzer 121, at least one quality assurance action on the analyzer 121.

The quality assurance may comprise ensuring one or more of reliability, confidence and credibility of measurement results within defined tolerances, e.g. that measurement results are valid with respect to one or more of traceability, accuracy, and precision. Specifically, the quality assurance may comprise blocking the generation of results in order not to generate results that maybe not reliable. The quality assurance may allow generating results but with attaching a flag to indicate that the result maybe not reliable.

The recall may be or may comprise a request from a manufacturer to return at least one item of the analyzer 121, e.g. after a discovery of malfunction and/or defects and/or safety issues.

The recalled item may be any item used for generating the measurement result. The recalled item may be any item which is administered in a database and/or software by using an identifier. The recalled item, for example, may be at least one item selected from the group comprising: at least one reagent; at least one diluent; at least one detergent; at least one consumable, e.g. pipette tip, assay cup or reaction vessel, or consumable rack or tray, at least one ion selective electrode, at least one spare part, assay reagent, diluent, detergent, system reagent, calibrator, control sample, assay application, calibrator application, control application, electrodes, software versions and the like.

The recall message may be or may comprise a computer readable file, e.g. of an arbitrary computer readable data format such as a xml file or HL7, comprising information about at least one recall, specifically about the identity of the recalled item. The information about an identity of a recalled item of the analyzer 121 may be any information allowing for unique identification of an item of the analyzer 121. The information about the identity of the recalled item may comprise one or more of at least one identifier, a code, a lot number, a version number, information about affected analyzer, information about a compartment ID, an affected software version, time limit, and information about an action to be applied.

The method for quality assurance will be described exemplarily in more detail in FIG. 2. FIG. 2 shows a flowchart of an exemplary embodiment of the method depicted in FIG. 1 according to the present invention.

Preceding the retrieving 110 at least one recall message 116, in the embodiment shown in FIG. 2, a recall message 116, e.g. as a recall file, may be published and/or deployed (denoted with reference number 118), e.g. by the manufacturer.

In the embodiment of FIG. 2, the recall file may comprise the following content: information about the affected analyzer, information about the affected analyzer compartment (AC) or module, a component ID, a lot number, a version number, information about an affected software version, time limit (e.g. after X hours), file expiration date, to be defined. In this example, information about an affected software version is given as information about measure (from 0 to 9), wherein the measure maps in the software to different workflows as a reaction to recall.

The recall message may be retrieved in step a) 110 by at least one of the following: downloading the recall message such as from at least one data storage device, from a web- or cloud-based data storage device; obtaining the recall message via at least one computer network, such as the Internet; obtaining the recall message via at least one wire-based and/or wireless interface. The retrieving 110 may fully or partially take place automatically, such as by automatic download of the recall message 116. The retrieving 110 may comprise, e.g. in addition, manual steps, e.g. as a part of a service/maintenance workflow such as inputting recall files. For example, the recall message 116 is retrieved via at least one communication interface 120 of the IVD analyzer 121. For example, the recall message 116 may be retrieved automatically, e.g. is retrieved automatically via the communication interface 120. For example, the retrieving 110 of the recall message 116 may be automatically executed at predefined time intervals. For example, the recall message 116 may be sent remotely to the analyzer. The predefined time intervals may be adapted to the routines of the system, e.g. synchronization to a repository may take place every 24 hours. No action may be required on the part of the customer/user.

The automatically receiving in step a) may comprise, as shown in FIG. 2, reading 122 the measure and the time limit, e.g. by using a processor of the IVD analyzer 121. Next, the status of the IVD analyzer 121 is checked 124, e.g. if the status is standby or not. If the system status is other than standby (System status other than standby “Yes”), the method may comprise displaying at least one information about the retrieving of the recall message 116 and/or about the recall message 116 via at least one user interface 126, e.g. at least one graphical user interface (GUI), e.g. to inform the user on the GUI about the receiving of a recall file. In the embodiment of FIG. 2, the user interface 126 exemplarily shows “Alarm: Recall file received, system will change to system stop or standby after XX hours” (wherein XX may depend on the information received by the recall message or may be defined by software executed by the processor and/or the communication interface). After interval is exceeded, the system status changes 128 to standby and autodownload of the recall file may be executed. If the system status is standby (System status other than standby “No”), the workflow may continue with 128. An automatic feedback is given to the communication interface 120 that the recall file 116 is installed on the IVD analyzer 121 (e.g. incl. serial number). In addition, further information may be provided as automatic feedback such as the file type (cdbx message, AU log for a data warehouse) and/or an upload frequency and the like.

In step b) 112, the recall message 116 is processed by determining if the recalled item is onboard the analyzer 121. The processing the recall message 116 may comprise at least one arbitrary action/process such as a workflow on the recall message 116 for determining if the recalled item is onboard the analyzer 121. The processing of the recall message may comprise searching with respect to the information about the identity of the recalled item in at least one database of the analyzer 121 and/or in at least one remote database. The database may be or may comprise at least one database selected from the group comprising: at least one server, at least one server system comprising a plurality of servers, at least one cloud server or cloud computing infrastructure. The database may comprise at least one storage unit configured to store data. The determining if the recalled item is onboard the analyzer 121 further may comprise determining if an installed software is affected.

In case the recalled item is determined not to be onboard the analyzer 121, operation of the analyzer may be continued and/or at least one information about the absence of the recalled item onboard the analyzer 121 may be displayed. For example, as exemplarily shown in FIG. 2, the user interface 126 may display “Alarm: recall published but this instrument is not affected”. In addition, as shown in FIG. 2, the user interface 126 may display “Alarm: Recalled item is not on board, Operation possible”. The operation 130 can be started.

However, in case the recalled item is determined to be onboard the analyzer (“YES”), in step c) at least one quality assurance action is applied on the analyzer 121. The quality assurance action may comprise at least one action having an impact on the quality assurance. FIG. 2, shows exemplarily five different quality assurance actions for different recalled items. For example, the quality assurance action may comprise determining if an assay reagent or diluent is affected 132 and if this is the case to automatically mask the affected assay 134. In addition, an alarm may be issued (e.g. “Alarm: recalled test is masked”). For example, the quality assurance action may comprise determining if a system reagent or detergent is affected 136 and if this is the case to automatically mask the AC/module 138. In addition, an alarm may be issued (e.g. “Alarm: AC/module is masked”). For example, the quality assurance action may comprise determining if an ion selective electrode (ISE electrode) or reagent is affected 140 and if this is the case to automatically mask the affected ISE AC/module/electrode 142. In addition, an alarm may be issued (e.g. “Alarm: AC/module is masked”). For example, the quality assurance action may comprise determining if an assay cup tray or Immuno Assay (IA) system reagent is affected 144 and if this is the case to automatically mask the affected IA AC/module 146. In addition, an alarm may be issued (e.g. “Alarm: AC/module is masked”). For example, the quality assurance action may comprise determining if a spare part is affected 148 and if this is the case to automatically mask the affected AC/module/system 150. In addition, an alarm may be issued (e.g. “Alarm: AC/module/system is masked”).

The method may comprise unblocking one or more of the recalled item, the affected unit, the affected module or the analyzer 121, when the recalled item is removed from the analyzer 121 and/or exchanged with a not affected one and/or after receiving a message indicating that the item can be used again such as after a software update.

FIG. 2 further shows a workflow in case a recalled item is registered at a later time point. The method may comprise detecting 152 that a recalled item is registered at a later time point. For example, in case a new item is registered, the method may comprise searching with respect to the identity of the new item in the database of the analyzer 121 and/or in the remote database and may determine if the new item is object to a recall. In this case 156, at least one quality assurance action may be applied. Otherwise 154, e.g. in case the new item is not determined to be object to a recall, operation of the analyzer 121 may be continued.

### LIST OF REFERENCE NUMBERS

110 retrieving at least one recall message
112 processing the recall message
114 applying at least one quality assurance action
116 recall message
118 published and/or deployed
120 communication interface
121 IVD analyzer
122 reading
124 checking
126 user interface
128 system status changes
130 operation can start
132 assay reagent or diluent is affected
134 automatically mask the affected assay
136 system reagent or detergent is affected
138 automatically mask the AC/module
140 ISE electrode or reagent is affected
142 automatically mask the affected ISE AC/module/electrode
144 assay cup tray or IA system reagent is affected
146 automatically mask the affected IA AC/module
148 spare part is affected
150 automatically mask the affected AC/module/system
152 detecting
154 new item is not determined to be object to a recall
156 the new item is object to a recall

1. A computer implemented method for quality assurance of an in vitro-diagnostic analyzer, the method comprising:
a) retrieving at least one recall message comprising information about an identity of a recalled item of the analyzer;
b) processing the recall message by determining if the recalled item is onboard the analyzer;
c) applying, in case the recalled item is determined to be onboard the analyzer, at least one quality assurance action on the analyzer,
wherein the quality assurance action comprises at least one action selected from the group comprising: preventing usage of the recalled item and/or related to the recalled item for further operation of the analyzer by one or more of automatically masking the recalled item in a control menu and/or preventing execution of software relating to the recalled item; blocking at least one affected module and/or at least one affected unit, wherein the method comprises unblocking one or more of the recalled item, the affected unit, the affected module or the analyzer, when the recalled item is re-moved from the analyzer and/or exchanged with a not affected one and/or after receiving a message indicating that the item can be used again.

2. The method according to claim 1, wherein the quality assurance action comprises at least one action selected from the group comprising:
displaying at least one information about presence of the recalled item onboard the analyzer; preventing operation of the analyzer; applying at least one action as defined in the recall message such as attaching a data alarm to every result which is produced by using the recalled item.

3. The method according to claim 1, wherein, in case the recalled item is determined not to be onboard the analyzer, operation of the analyzer is continued and/or at least one information about the absence of the recalled item onboard the analyzer is displayed.

4. The method according to claim 1, wherein the recalled item is at least one item selected from the group comprising: at least one reagent; at least one diluent; at least one detergent; at least one consumable, e.g. pipette tip, assay cup or reaction vessel, or consumable rack or tray, at least one ion selective electrode, at least one spare part, assay reagent, diluent, detergent, system reagent, calibrator, control sample, assay application, calibrator application, control application, electrodes, software versions and the like.

5. The method according to claim 1, wherein the recall message is retrieved automatically, wherein the retrieving of the recall message is automatically executed at predefined time intervals, and/or wherein the recall message is sent remotely to the analyzer.

6. The method according to claim 1, wherein the method comprises displaying at least one information about the retrieving of the recall message and/or about the recall message via at least one user interface.

7. The method according to claim 1, wherein the information about the identity of the recalled item comprises one or more of at least one identifier, a code, a lot number, a version number, information about affected analyzer, information about a compartment ID, an affected software version, time limit, and information about an action to be applied.

8. The method according to claim 1, wherein the determining if the recalled item is onboard the analyzer comprises searching with respect to the information about the identity of the recalled item in at least one database of the analyzer and/or in at least one remote database.

9. The method according to claim 1, wherein the determining if the recalled item is onboard the analyzer comprises determining if an installed software is affected.

10. An in vitro-diagnostic analyzer for analyzing at least one sample, the analyzer comprises
at least one communication interface configured for retrieving at least one recall message comprising information about an identity of a recalled item of the analyzer;
at least one processor configured for processing the recall message by determining if the recalled item is onboard the analyzer;
at least one control unit configured for applying, in case the recalled item is determined to be onboard the analyzer, at least one quality assurance action on the analyzer, wherein the quality assurance action comprises at least one action selected from the group comprising: preventing usage of the recalled item and/or related to the recalled item for further operation of the analyzer by one or more of automatically masking the recalled item in a control menu and/or preventing execution of software relating to the recalled item; blocking at least one affected module and/or at least one affected unit, wherein the in vitro-diagnostic analyzer is configured for unblocking one or more of the recalled item, the affected unit, the affected module or the analyzer, when the recalled item is re-moved from the analyzer and/or exchanged with a not affected one and/or after receiving a message indicating that the item can be used again.

11. The analyzer according to claim 10, wherein the analyzer is configured for performing the method comprising
a) retrieving at least one recall message comprising information about an identity of a recalled item of the analyzer;
b) processing the recall message by determining if the recalled item is onboard the analyzer;
c) applying, in case the recalled item is determined to be onboard the analyzer, at least one quality assurance action on the analyzer,
wherein the quality assurance action comprises at least one action selected from the group comprising: preventing usage of the recalled item and/or related to the recalled item for further operation of the analyzer by one or more of automatically masking the recalled item in a control menu and/or preventing execution of software relating to the recalled item; blocking at least one affected module and/or at least one affected unit, wherein the method comprises unblocking one or more of the recalled item, the affected unit, the affected module or the analyzer, when the recalled item is re-moved from the analyzer and/or exchanged with a not affected one and/or after receiving a message indicating that the item can be used again.

12. A computer program comprising instructions which, when the program is executed by an in vitro-diagnostic analyzer according to claim 10, cause the analyzer to perform a method comprising
a) retrieving at least one recall message comprising information about an identity of a recalled item of the analyzer;
b) processing the recall message by determining if the recalled item is onboard the analyzer;
c) applying, in case the recalled item is determined to be onboard the analyzer, at least one quality assurance action on the analyzer,
wherein the quality assurance action comprises at least one action selected from the group comprising: preventing usage of the recalled item and/or related to the recalled item for further operation of the analyzer by one or more of automatically masking the recalled item in a control menu and/or preventing execution of software relating to the recalled item; blocking at least one affected module and/or at least one affected unit, wherein the method comprises unblocking one or more of the recalled item, the affected unit, the affected module or the analyzer, when the recalled item is re-moved from the analyzer and/or exchanged with a not affected one and/or after receiving a message indicating that the item can be used again.

13. A computer-readable storage medium comprising instructions which, when the instructions are executed by the an in vitro-diagnostic analyzer according to claim 10, cause the analyzer to perform a method comprising
a) retrieving at least one recall message comprising information about an identity of a recalled item of the analyzer;
b) processing the recall message by determining if the recalled item is onboard the analyzer;
c) applying, in case the recalled item is determined to be onboard the analyzer, at least one quality assurance action on the analyzer,
wherein the quality assurance action comprises at least one action selected from the group comprising: preventing usage of the recalled item and/or related to the recalled item for further operation of the analyzer by one or more of automatically masking the recalled item in a control menu and/or preventing execution of software relating to the recalled item; blocking at least one affected module and/or at least one affected unit, wherein the method comprises unblocking one or more of the recalled item, the affected unit, the affected module or the analyzer, when the recalled item is re-moved from the analyzer and/or exchanged with a not affected one and/or after receiving a message indicating that the item can be used again.

14. A non-transient computer-readable medium including instructions that, when executed by one or more processors, cause the one or more processors to perform the method according to claim 1.
